(19) United States
(12) Patent Application Publication
(10) Pub. No.: US 2013/0096609 A1
(43) Pub. Date: Apr. 18, 2013

(54) Tube-Type Medical Needle Unit And Method Of Inserting Plurality Of Medical Sutures Into Tube-Type Medical Needle

(71) Applicants: Heeyoung Lee, Seoul (KR); Hyunjin Yang, Seoul (KR)

(72) Inventors: Heeyoung Lee, Seoul (KR); Hyunjin Yang, Seoul (KR)

(21) Appl. No.: 13/807,003

(22) PCT Filed: Jun. 20, 2011
(86) PCT No.: PCT/KR2011/004469

§ 371 Date: Dec. 26, 2012

(30) Foreign Application Priority Data
Jun. 25, 2010 (KR) 10-2010-0060666

(51) Int. Cl. A61B 17/06

(52) U.S. Cl. CPC A61B 17/06066
USPC 606/223; 29/428

(57) ABSTRACT

Provided are a tube-type medical needle unit and a method of mounting a plurality of medical sutures in a tube-type needle. The tube-type medical needle unit includes a tube-type needle including an end in the shape of a probe and including a communication hole formed therein to communicate the end with another end and a plurality of sutures inserted into the communication hole of the tube-type needle, the sutures including adhesion protrusions formed on surfaces thereof, in which the plurality of sutures are arranged in parallel and mounted in the tube-type needle.

### TECHNICAL FIELD

The present invention relates to a tube-type medical needle unit and a method of inserting a plurality of medical sutures into a tube-type medical needle, and more particularly, to a tube-type medical needle unit which makes it simple and easy to perform an operation and allows a plurality of sutures to have a sufficient support force in a human body during adhesion and a method of inserting a plurality of medical sutures into a tube-type medical needle of the tube-type medical needle unit.

### BACKGROUND ART

Recently, in a way to perform an operation for removing wrinkles from the skin of a human body, a suture, that is, thread safe for the human body (authorized for use in human bodies during an operation), is inserted under the skin and adheres subcutaneous tissues with dermis tissues, thus strengthening tension. If the adhered parts are pulled and fixed upward, some loose part moves up and is fixed through the tension of the suture.

The suture used in the operation is processed to have an adhesion protrusion such as a thorn or a leaf, thereby an adhesive force with respect to body tissues, and moreover, directivity is applied to the thorn such that the suture is caught by some tissue and fixed in a certain direction. In addition, there have been various attempts to reinforce the adhesion force, for example, by varying the directivity.

Once the suture is attached or adhered into the human body, the other side of the suture is pulled in a desired direction and is fixed to another part of the human body, thus flattening wrinkles on the skin or moving a part of the human body. The operation using the suture, when compared to other operations for achieving similar objects, minimizes incising or cutting the human body, reduces an operation time, intends to simplify the operation, and reduces swelling and a recovery period. Due to such advantages, the operation using the suture has been remarkably developed in recent times.

In the wrinkle removing operation using the suture, the suture should have sufficient adhesion strength and the number of pokes of a needle for adhering the suture into the skin has to be minimized.

When the suture has sufficient adhesion strength, it can keep the skin firm for a long period of time. By reducing the number of pokes of the needle, damage to the human body can be minimized, thus shortening the recovery time and minimizing undesired side effects.

### DISCLOSURE OF INVENTION

### Technical Problem

However, a single piece of thread has been inserted into a single needle for an operation so far. That is, as shown in FIG. 1, a single suture 100 is inserted into a desired portion on the human body's skin. The suture 100 can be easily adhered to the tissue by means of adhesion protrusions 101 formed on the outer surface of the suture 100. As such, when the single suture 100 is inserted into the desired portion, a sufficient support force cannot be obtained due to a weak support force of the suture 100. Although a sufficient support force may be obtained by insertion of a thick suture, the thick suture has low elasticity and is relatively hard, thus being limited to particular purposes. Moreover, the suture inserted into the skin may be noticeable from outside, which may spoil the beauty.

To solve the problem, several sutures may be separately inserted into a desired portion through several pokes. However, due to adhesion protrusions of the previously inserted suture, the new suture is difficult to be installed close to the previously inserted suture. That is, the new suture has to be disposed into the skin with a predetermined interval with the previously inserted suture, making it difficult to obtain a desired result.

Moreover, to insert several sutures, the needle has to be poked into the skin several times. In this case, the several pokes of the needle may increase damage to the skin, lengthening the recovery time after an operation and increasing the probability of a secondary infection due to excessive skin damage.

In a common eye needle in which an eye of the needle is formed only in a rear portion to thread the needle, plural pieces of thread composed of gold thread and a suture are mounted in parallel in the eye. Such a needle may be used to remove wrinkles, but it is not a tube-type needle having a communication hole which communicates one end with the other end, such that the plural pieces of thread have to be inserted into the body along a path along which the needle has moved. Therefore, there exist both a portion poked by the needle and a portion from which the needle perforates out in the human body in the operation. However, it is not easy to select a proper position of the needle-perforated portion according to a needle-inserted direction, making it difficult to perform the operation.

Moreover, the suture, when being attached to the eye of the needle, moves through the body along the needle. In this process, the suture moves in a state of being exposed to the body and thus, friction is generated by contact between the suture and the surface of the body, making it difficult to perform the operation.

### Solution To Problem

The present invention provides a tube-type medical needle unit, in which a number of sutures are inserted into a human body in a single needle poking process, thus strengthening a support force based on adhesion and minimizing damage to the human body, and a method of mounting a plurality of medical sutures into a tube-type needle of the tube-type medical needle unit.

The present invention also provides a tube-type medical needle unit, in which when a plurality of sutures are mounted by passing through a medicine injection-type needle, that is, a tube-type needle, an advancing front end of the needle and front ends of the sutures are placed on similar positions and then the needle is inserted into a body, thus reducing friction with body tissues which may be incurred during movement of the sutures having protrusions through the body and facilitating the overall operation through removal of a need for a portion of the body from which the needle perforates out, and a method of mounting a plurality of medical sutures into a tube-type needle of the tube-type medical needle unit.

According to an aspect of the present invention, there is provided a tube-type medical needle unit includes a tube-type needle including an end in the shape of a probe and including a communication hole formed therein to communicate the end with another end and a plurality of sutures inserted into the communication hole of the tube-type needle, the sutures including adhesion protrusions formed on surfaces thereof, in which the plurality of sutures are arranged in parallel and mounted in the tube-type needle.

A cluster portion formed by clustering the plurality of sutures may be formed in at least a position.

The at least a position where the cluster portion is formed may include end portions of the plurality of sutures adjacent to an end portion of the tube-type needle.

The plurality of sutures each may be composed of any one of a group consisting of nylon, polypropylene, polydioxanon, polycarprolacton, poly-L-lactic acid (PLLA), polyglycolic acid (PGA), poly-lactic-glyolic acid(PLGA), cat gut, gold(Au), an alloy including Au, platinum(Pt), an alloy including Pt, Titanium(Ti), and an alloy including Ti.

According to another aspect of the present invention, there is provided a method of mounting a plurality of sutures in a tube-type needle which includes an end in the shape of a probe and includes a communication hole formed therein to communicate the end with another end, the method including a clustering operation of clustering end portions of the plurality of sutures to form a cluster portion and a suture mounting operation of first inserting the cluster portion of the plurality of sutures into the communication hole through the another end of the tube-type needle and then inserting the other part of the sutures than the cluster portion in succession from the cluster portion into the communication hole.

The method may further include, after the suture mounting operation, a cluster portion extracting operation of extracting the cluster portion of the sutures through the end of the tube-type needle and a cluster portion cutting operation of cutting the extracted cluster portion.

In the clustering operation, the cluster portion may be formed by twist.

In the clustering operation, the cluster portion may be formed by knotting.

In the clustering operation, the cluster portion may be formed by thermal fusion.

In the clustering operation, the cluster portion may be formed by bending and folding a single suture.

### Advantageous Effects of Invention

The tube-type medical needle unit according to the present invention can provide a sufficient support force because the plurality of sutures are fixed to the fixed portion. In particular, a sufficient support force can be secured without increasing the thicknesses of the sutures, thereby preventing a trace of the sutures from leaving on the skin.

Moreover, since the tube-type medical needle unit according to the present invention includes the plurality of sutures in the single tube-type needle, a plurality of pokes of the needle are not required for inserting the plurality of sutures into tissues, thereby preventing excessive damage to the tissues and thus shortening the recovery time.

Furthermore, when the plurality of sutures are inserted into tissues, those tissues are adhered between the sutures, allowing the sutures to surely have a fixing force in the tissues.

### BRIEF DESCRIPTION OF DRAWINGS

The above and other features and advantages of the present invention will become more apparent by describing in detail exemplary embodiments thereof with reference to the attached drawings in which:

FIG. 1 is a diagram showing an example using a tube-type medical needle unit according to prior art;

FIG. 2 is a diagram showing an example using a tube-type medical needle unit according to an exemplary embodiment of the present invention;

FIG. 3 is a diagram showing a state after the tube-type medical needle unit shown in FIG. 2 is used;

FIG. 4 is a cross-sectional view of the tube-type medical needle unit shown in FIG. 2;

FIG. 5 is a diagram showing a method of inserting a plurality of medical sutures into the tube-type medical needle unit shown in FIG. 4;

FIG. 6 is a cross-sectional view of a tube-type medical needle unit according to another exemplary embodiment of the present invention;

FIG. 7 is a cross-sectional view of a tube-type medical needle unit according to another exemplary embodiment of the present invention; and

FIG. 8 is a cross-sectional view of a tube-type medical needle unit according to another exemplary embodiment of the present invention.

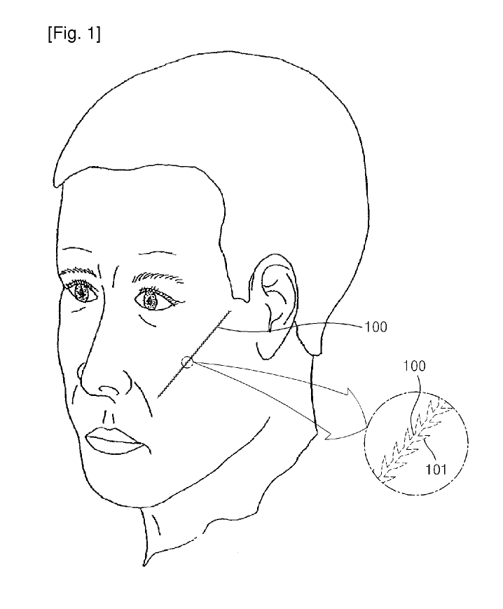

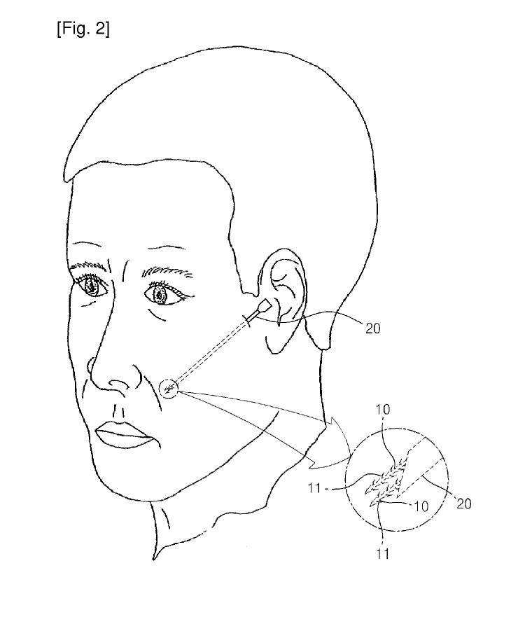

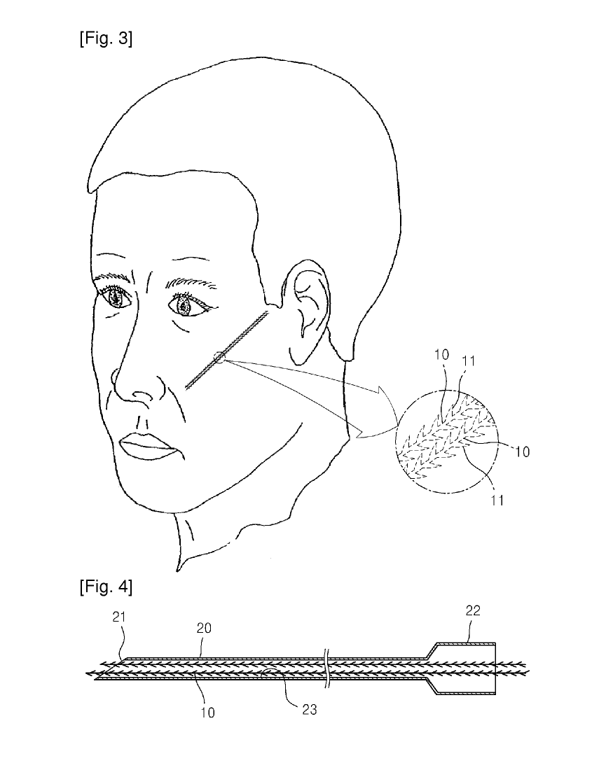

### MODE FOR THE INVENTION

Hereinafter, a tube-type medical needle unit according to exemplary embodiments of the present invention will be described in detail with reference to the accompanying drawings.

A tube-type medical needle unit 1 according to an exemplary embodiment of the present invention includes a tube-type needle 20 and sutures 10.

The tube-type needle 20 has an end 21 in the shape of a probe and has a communication hole 23 therein in its longitudinal direction to communicate the end 21 with another end 22. The tube-type needle 20 may be inserted into a human body through the end 21, and has mounted the sutures 10 therein.

The tube-type needle 20 is preferably made of a metal material, but may be made of various materials without being limited to the metal material, as long as they have high rigidity and are not harmful to human bodies.

The another end 22 of the tube-type needle 20 has a large-diameter portion to allow the sutures 10 to be easily inserted into the tube-type needle 20.

The sutures 10 are inserted and mounted into the communication hole 23 of the tube-type needle 20. More specifically, the sutures 10 have an overall shape of thread and have, on surfaces thereof, a plurality of adhesion protrusions 11 in the form of thorns or leaves. The adhesion protrusions 11 are intended to strengthen adhesion strength with respect to human body's tissues, and preferably, may have directivity to be caught and fixed by the tissues in a single direction.

The directivity of the adhesion protrusions 11 may be not only unidirectional, but also bi-directional. For example, the adhesion protrusions 11 may have unidirectional directivity in some sections, and may have bidirectional directivity in some other sections. In this way, various design changes may be possible according to operation conditions.

In the current embodiment, unlike in prior art, the plurality of sutures 10 are arranged in parallel within the tube-type needle 20 and thus are mounted within the tube-type needle 20. As such, when the plurality of sutures 10 are arranged in parallel, the plurality of sutures 10 can be adhered to tissues by a single poke of the tube-type needle 20.

The sutures 10 may use various materials to maximize adhesion strength with human body tissues, improve elasticity, and strengthen support force, such as nylon, polypropylene, polydioxanon, polycarprolacton, poly-L-lactic acid (PLLA), polyglycolic acid (PGA), poly-lactic-glyolic acid(PLGA), cat gut, gold(Au), an alloy including Au, platinum(Pt), an alloy including Pt, Titanium(Ti), an alloy including Ti, and so forth.

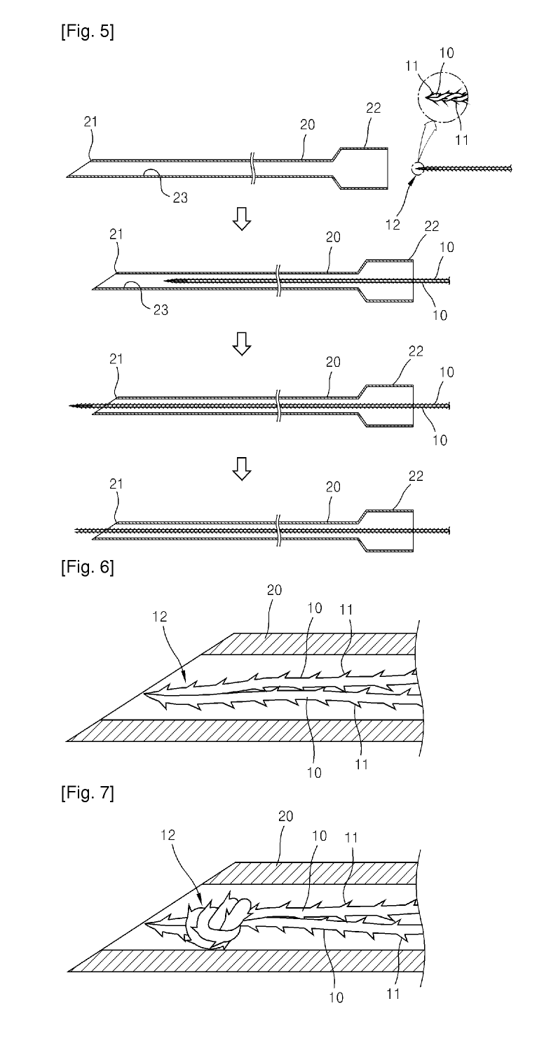

A method of mounting the plurality of sutures 10 according to the present invention into the tube-type needle 20 will be described in detail with reference to FIG. 5.

More specifically, the method includes a clustering operation, a suture mounting operation, a cluster portion extracting operation, and a cluster portion cutting operation.

In the clustering operation, prior to insertion of the plurality of sutures 10 into the tube-type needle 20, the plurality of sutures 10 are arranged to be easily inserted into the tube-type needle 20. More specifically, the plurality of sutures 10 are arranged in parallel and end portions of the respective sutures 10 are disposed at the same position, after which the end portions of the sutures 10 are clustered to form a cluster portion 12.

In the clustering operation, the cluster portion 12 is formed by twisting the end portions of the sutures 10 together as shown in an enlarged view of FIG. 5.

In the suture mounting operation, the plurality of sutures 10 having the cluster portion 12 are inserted into the communication hole 23 of the tube-type needle 20. More specifically, the cluster portion 21 of the plurality of sutures 10 is first inserted into the communication hole 23 through the another end 22 of the tube-type needle 20 which has the probe-shaped end 21 and the communication hole 23 for communicating the end 21 with the another end 22. Thereafter, the plurality of sutures 10 are further inserted into the communication hole 23, such that the plurality of sutures 10 can be sufficiently inserted into the tube-type needle 20.

More specifically, after insertion of the cluster portion 12, the other part of the sutures 10 is inserted in succession from the cluster portion 12 into the communication hole 23, such that the sutures 10 are arranged in parallel in the communication hole 23.

In the cluster portion extracting operation, the cluster portion 12 of the sutures 10 arranged in the tube-type needle 20 is extracted through the end 21 of the tube-type needle 20. That is, the plurality of sutures 10 disposed in the communication hole 23 through the suture mounting operation are extracted such that the plurality of sutures 10 can be sufficiently arranged in parallel.

In the cluster portion cutting operation, the extracted cluster portion 12 is cut. More specifically, the vicinity of the cluster portion 12 is cut using a predetermined cutter to separate the clustered sutures 10.

The tube-type medical needle unit manufactured as described above can be used as described below.

First, after a predetermined cutting portion is formed on a patient's face, the tube-type needle 20 having the sutures 10 mounted therein is inserted between subcutaneous tissues and dermis tissues. After the tube-type needle 20 is inserted into a desired fixed portion, the plurality of sutures 10 are adhered to the fixed portion. After the tube-type needle 20 is removed and then the sutures 10 are pulled and fixed to another portion of the human body, thus smoothing wrinkles.

The tube-type medical needle unit according to the present invention can provide a sufficient support force because the plurality of sutures are fixed to the fixed portion. In particular, a sufficient support force can be secured without increasing the thicknesses of the sutures, thereby preventing a trace of the sutures from leaving on the skin.

Moreover, since the tube-type medical needle unit according to the present invention includes the plurality of sutures in the single tube-type needle, a plurality of pokes of the needle are not required for inserting the plurality of sutures into tissues, thereby preventing excessive damage to the tissues and thus shortening the recovery time.

Furthermore, when the plurality of sutures are inserted into tissues, those tissues are adhered between the sutures, allowing the sutures to surely have a fixing force in the tissues.

The tube-type medical needle unit according to the present invention can be manufactured or modified as described below.

While it has been described that the cluster portion 12 is formed by twist, but the cluster portion 12 may also be formed by thermal fusion as shown in FIG. 6 or by knotting as shown in FIG. 7.

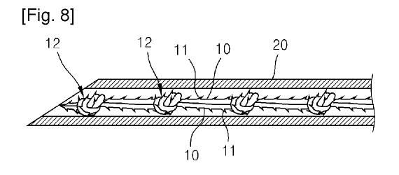

The cluster portion 12 clustering the plurality of sutures 10 may be formed in at least a position. That is, the cluster portion 12 may be disposed in a plurality of positions as shown in FIG. 8, and the cluster portion 12 may be formed in the end portions of the sutures 10 which are adjacent to the end portion of the tube-type needle 20 as shown in FIGS. 6 and 7.

The cluster portion 12 allows the sutures 10, when inserted into the body, to easily sit in a desired position.

In addition, the method of forming the cluster portion 12 is not limited to the foregoing methods, and the cluster portion 12 may also be formed by bending and folding a single suture. For example, with respect to an approximately center portion of the single suture, protrusions having unidirectional directivity are formed at a side of the center portion and protrusions having the opposite directivity are formed at the other side of the center portion, such that the suture is folded with respect to the center portion, thus forming the cluster portion 12. The folded center portion is the cluster portion 12. Meanwhile, the folded center portion may be heated to maintain the folded state.

In the tube-type medical needle unit according to the present invention, since the plurality of sutures are arranged in parallel in the tube-type needle, the plurality of sutures in a state of being adjacent to each other are inserted and adhered into the skin through a single poke of the tube-type needle, such that the sutures can have a sufficient support force.

Moreover, there is no need for a plurality of pokes of the tube-type needle to insert the plurality of sutures, minimizing damage to the body.

Furthermore, the needle unit according to the present invention is of a tube type and has the plurality of sutures therein, thereby preventing unnecessary friction from being generated during movement of the sutures, and removing a need to consider a portion of the body from which the tube-type needle perforates out and thus facilitating the overall operation.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A tube-type medical needle unit comprising:
a tube-type needle comprising an end in the shape of a probe and comprising a communication hole formed therein to communicate the end with another end; and
a plurality of sutures inserted into the communication hole of the tube-type needle, the sutures comprising adhesion protrusions formed on surfaces thereof,
wherein the plurality of sutures are arranged in parallel and mounted in the tube-type needle.

2. The tube-type medical needle unit of claim 1, wherein a cluster portion formed by clustering the plurality of sutures is formed in at least a position.

3. The tube-type medical needle unit of claim 2, wherein the at least a position where the cluster portion is formed comprises end portions of the plurality of sutures adjacent to an end portion of the tube-type needle.

4. The tube-type medical needle unit of claim 2, wherein the at least a position where the cluster portion is formed comprises end portions of the plurality of sutures adjacent to an end portion of the tube-type needle.

5. A method of mounting a plurality of sutures in a tube-type needle which comprises an end in the shape of a probe and comprises a communication hole formed therein to communicate the end with another end, the method comprising:
a clustering operation of clustering end portions of the plurality of sutures to form a cluster portion; and
a suture mounting operation of first inserting the cluster portion of the plurality of sutures into the communication hole through the another end of the tube-type needle and then inserting the other part of the sutures than the cluster portion in succession from the cluster portion into the communication hole.

6. The method of claim 5, further comprising, after the suture mounting operation:
a cluster portion extracting operation of extracting the cluster portion of the sutures through the end of the tube-type needle; and
a cluster portion cutting operation of cutting the extracted cluster portion.

7. The method of claim 5, wherein in the clustering operation, the cluster portion is formed by twist.

8. The method of claim 5, wherein in the clustering operation, the cluster portion is formed by knotting.

9. The method of claim 5, wherein in the clustering operation, the cluster portion is formed by thermal fusion.

10. The method of claim 5, wherein in the clustering operation, the cluster portion is formed by bending and folding a single suture.

11. The method of claim 6 wherein in the clustering operation, the cluster portion is formed by twist.

12. The method of claim 6 wherein in the clustering operation, the cluster portion is formed by knotting.

13. The method of claim 6 wherein in the clustering operation, the cluster portion is formed by thermal fusion.

14. The method of claim 6 wherein in the clustering operation, the cluster portion is formed by bending and folding a single suture.
